(19) United States
(12) Patent Application Publication
(10) Pub. No.: US 2005/0069511 A1
(43) Pub. Date: Mar. 31, 2005

(54) Cosmetic compositions containing a starch and an ester and the use thereof

(76) Inventors: Armelle Magnet, Irigny (FR); Sandrine Decoster, Saint Gratien (FR); Laurent Chesneau, Levallois Perret (FR)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/478,167

(22) PCT Filed: May 17, 2002
(86) PCT No.: PCT/FR02/01686

(30) Foreign Application Priority Data
May 18, 2001 (FR) 01/06603

(51) Int. Cl. A61K 7/06; A61K 7/11

(52) U.S. Cl. 424/70.13

(57) ABSTRACT

The present invention relates to a cosmetic composition comprising: a cosmetically acceptable medium, at least one starch and at least one ester selected from (1) liquid esters from a C3-C30 carboxylic acid and a C1-C30 alcohol, with at least one from the acid or the alcohol is branched or possesses at least one carbon-carbon double bond; and (2) esters from a C7-C30 aromatic acid having a carboxylic group that is directly linked to the aromatic ring, and a C1-C30 alcohol. The inventive composition is substantially free of soap. This composition has a “melting” texture and can be easily rinsed off. Hair treated with this composition has a soft feel with no residues. The inventive composition is preferably suitable for washing and/or conditioning keratinous materials, such as hair or skin.

### BACKGROUND OF THE INVENTION

The present invention relates to new cosmetic compositions comprising: a cosmetically acceptable medium, at least one specific carboxylic ester, and at least one starch.

It is well known that hair which has been sensitized (i.e., damaged and/or embrittled) to various degrees under the action of atmospheric agents or under the action of mechanical or chemical treatments, such as coloring, bleaching and/or perming, is often difficult to disentangle and to style, and lacks softness.

Proposals have already been made for the treatment of keratin materials ,for example, hair with cosmetic compositions containing thickening polysaccharides such as, starch or celluloses.

Such compositions, however, have drawbacks such as problems of rinsability, problems of stability to acidic pH, difficulties in distribution on the keratin materials, and inadequate cosmetic properties.

Recommendations have already been made to use cationic polymers in compositions for washing or caring for keratin materials such as the hair in order to facilitate the disentangling of the hair and to impart to it softness and suppleness. The use of cationic polymers for this purpose presents a variety of drawbacks. Owing to their high affinity for the hair, some of these polymers are deposited to a substantial extent in the course of repeated uses, and lead to undesirable effects such as an unpleasant heavy feel, a stiffening of the hair, and an interfiber adhesion which impacts styling.

Proposals have already been made to use oils such as vegetable or animal oils or else fatty acid esters as a conditioner. However, the keratin materials treated with these compositions usually have a critical greasy feel.

In summary, it is found that state of the art cosmetic compositions do not give complete satisfaction.

### SUMMARY OF THE INVENTION

The applicant has now found that the combination of a starch with a specific carboxylic ester allows these drawbacks to be remedied.

Consequently, following the major research efforts, it has now been found by the applicant that the use of compositions, preferably haircare compositions, based on a specific ester and a starch allows the problems set out above to be limited or even suppressed. The hair is easy to disentangle and is smooth from root to tip, with an improvement in the hold of the style.

Moreover, this combination imparts a “melting” texture to the cosmetic compositions: that is, a texture which disappears rapidly into the hair. Hair treated with this composition has a soft feel with no residues.

Moreover, the compositions of the invention, when applied to the skin, for example, in the form of a foam bath or shower gel, provide an improvement in the softness of the skin.

One aspect of the present invention provides new cosmetic compositions comprising:
a cosmetically acceptable aqueous medium; at least one starch;
at least one carboxylic ester in an amount of up to 10% based on the weight of the cosmetic composition wherein the carboxylic ester is selected from
1) liquid esters of a C3-C30 carboxylic acid and a C1-C30 alcohol, wherein at least one of the acid or the alcohol is branched or possesses at least one carbon-carbon double bond, or
2) esters of a C7-C30 aromatic acid and a C1-C30 alcohol, wherein the aromatic acid has carboxyl group directly bonded to the aromatic acid's ring;, at least 30% by weight of water relative to the total weight of the composition; and up to 20% by weight of fatty phase relative to the total weight of the composition,
   with the provisos that the composition is substantially free from fatty acid soap, the composition is free of nonsilicone cationic polymer if the starch is a starch phosphate, and, the carboxylic ester is not isopropyl palmitate or diisopropyl adipate, if the composition comprises a phosphorylated starch derivative, at least one thickener, and less than 3% by weight of surfactant.

Second aspect of the present invention provides new cosmetic compositions comprising:
a cosmetically acceptable aqueous medium; at least one starch;
at least one carboxylic ester in an amount of up to 10% based on the weight of the cosmetic composition wherein the carboxylic ester is selected from
1) liquid esters of a C3-C30 carboxylic acid and a C1-C30 alcohol, wherein at least one of the acid or the alcohol is branched or possesses at least one carbon-carbon double bond, or
2) esters of a C7-C30 aromatic acid and a C1-C30 alcohol, wherein the aromatic acid has carboxyl group directly bonded to the aromatic acid's ring; at least 30% by weight of water relative to the total weight of the composition; and up to 20% by weight of fatty phase relative to the total weight of the composition,
   with the provisos that the composition is substantially free from fatty acid soap, the composition is free of nonsilicone cationic polymer if the starch is a starch phosphate, and, the carboxylic ester is not isopropyl palmitate or diisopropyl adipate, if the composition comprises at least one thickener, and less than 3% by weight of surfactant.

Third aspect of the invention provides for a method of washing or caring for keratinous materials by applying the cosmetic composition defined above to keratinous materials.

Fourth aspect of the invention provides for a method of treating or conditioning keratinous materials by applying the cosmetic composition defined above to keratinous materials.

### DETAILED DESCRIPTION OF THE INVENTION

The different subject matter of the invention will now be detailed. All of the meanings and definitions of the compounds used in the present invention that are given below are valid for all of the subject matter of the invention.

Fatty acid soaps for the purposes of the invention are an alkali metal or alkaline earth metal or fatty amine salt of a C10-C18 fatty acid, stearic acid and isostearic acid.

“Substantially free from fatty acid soaps” means for the purposes of the invention that the concentration of fatty acid soaps is less than or equal to approximately 1.5% by weight, preferably less than approximately 0.5% by weight, and more preferably less than 0.1% by weight relative to the total weight of the composition.

The fatty phase comprises all fats which are insoluble in water at ambient temperature of the composition, such as, fatty esters, vegetable oils, mineral oils, synthetic oils, fatty alcohols, fatty acids, waxes, and silicones. The fatty phase represents preferably from 0.001 to 15% by weight and more preferably from 0.01 to 10% by weight relative to the total weight of the composition and more preferably still from 0.5 to 8% by weight.

The composition according to the invention contains less than 10% by weight of fatty esters. By fatty esters are meant the esters according to the invention and esters of a C3-C30 carboxylic acid and a C1-C30 alcohol which do not have at least one of the acid or the alcohol branched or possess at least one carbon-carbon double bond.

The starches which can be used in the present invention are more preferably macromolecules in the form of polymers composed of elementary units which are anhydroglucose units. The number of these units and their assembly permits a distinction to be made between amylose (linear polymer) and amylopectin (branched polymer). The relative proportions of amylose and of amylopectin, and their degree of polymerization, vary as a function of the botanical origin of the starches.

The starch molecules used in the present invention may originate botanically from cereals or else from root crops. Accordingly the starches are selected for example from corn starch, rice starch, manioc starch, tapioca starch, barley starch, potato starch, wheat starch, sorghum starch, or pea starch.

The starches are generally in the form of a white powder which is insoluble in cold water and whose elementary particles have a size ranging from 3 to 100 microns.

The starches used in the composition of the invention can be modified by one or more of the following reactions: pregelatinization, oxidation, crosslinking, esterification, and/or thermal treatments.

For example, these reactions may be carried out as follows:
pregelatinization by bursting the starch granules (for example, drying and cooking in a drying drum);
oxidation with strong oxidizing agents, resulting in the introduction of carboxyl groups into the starch molecule and in the depolymerization of the starch molecule (for example, by treating an aqueous starch solution with sodium hypochlorite);
crosslinking with functional agents capable of reacting with the hydroxyl groups of the starch molecules, which will thereby be bonded to one another (for example, with glyceryl and/or phosphate groups);
esterification in an alkaline medium for the grafting of functional groups, preferably C1-C6 acyl (acetyl) groups, C1-C6 hydroxyalkyl (hydroxyethyl, hydroxypropyl) groups, carboxymethyl groups, and octenylsuccinic groups. By crosslinking with phosphorus compounds it is possible, for example, to obtain monostarch phosphates (of the type St-O—PO—(OX)₂), distarch phosphates (of the type St-O—PO—(OX)—O-St) or even tristarch phosphates (of the type St-O—PO—(O-St)₂) or mixtures thereof.

X denotes, preferably, alkali metals (for example sodium or potassium), alkaline-earth metals (for example, calcium, magnesium), ammonium salts, amine salts such as those of monoethanolamine, diethanolamine, triethanolamine, 3-aminopropane-1,2-diol, or ammonium salts obtained from basic amino acids such as lysine, arginine, sarcosine, ornithine and citrulline.

The phosphorus compounds may be, for example, sodium tripolyphosphate, sodium orthophosphate, phosphorus oxychloride or sodium trimetaphosphate.

Preference will be given to using distarch phosphates or compounds rich in distarch phosphate, such as the product proposed under the references PREJEL VA-70-T AGGL (gelatinized hydroxypropylated manioc distarch phosphate) or PREJEL TK1 (gelatinized manioc distarch phosphate) or PREJEL 200 (gelatinized acetylated manioc distarch phosphate) by the AVEBE Company or STRUCTURE ZEA from NATIONAL STARCH (gelatinized corn distarch phosphate).

In accordance with the invention it is also possible to use amphoteric starches; these amphoteric starches contain one or more anionic groups and one or more cationic groups. The anionic and cationic groups can be bonded to the same reactive site of the starch molecule or to different reactive sites; preferably they are bonded to the same reactive site. The anionic groups may be of carboxylic, phosphate or sulfate type, preferably carboxylic. The cationic groups may be of primary, secondary, tertiary or quaternary amine type. The amphoteric starches are selected preferably from the compounds of the following formulae: 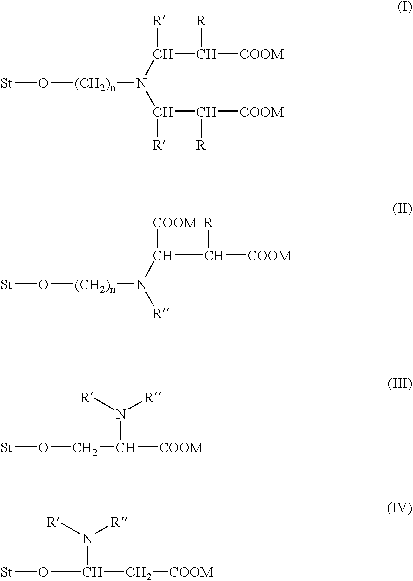
in which:
St-O represents a starch molecule,
R, identical or different, represents a hydrogen atom or a methyl radical,
R′, identical or different, represents a hydrogen atom, a methyl radical or a —COOH group,
n is an integer 2 or 3,
M, identical or different, denotes a hydrogen atom, an alkali metal or an alkaline-earth metal such as Na, K, Li, NH₄, a quaternary ammonium or an organic amine, and
R″ represents a hydrogen atom or an alkyl radical having from 1 to 18 carbon atoms.

These compounds are described in patents U.S. Pat. No. 5,455,340 and U.S. Pat. No. 4,017,460, which are included by reference. The starch molecules may be obtained from any of the plant sources of starch, such as, corn, potato, oats, rice, tapioca, sorghum, barley or wheat. It is also possible to use the hydrolyzates of the abovementioned starches. The starch is preferably obtained from potato.

The starches of formula (I) or (II) can be used according to the invention. More preferably used are starches modified with 2-chloroethylaminodipropionic acid, i.e., the starches of formula (I) or (II) in which R, R′, R″ and M represent a hydrogen atom and n is 2.

The starch or starches are used preferably in an amount ranging from 0.01 to 20% by weight relative to the total weight of the composition. More preferably this amount ranges from 0.05 to 15% by weight relative to the total weight of the composition, and more preferably still from 0.1 to 10% by weight.

The esters according to the invention are preferably liquid esters which are insoluble in water. The water-insoluble liquid carboxylic esters are insoluble in water at a concentration at or above 0.1% by weight in water at 25° C.; that is to say that, under these conditions, they do not form a transparent, macroscopically isotropic solution.

The total carbon number of the esters of the invention is generally greater than or equal to 10 and preferably less than 100 and more preferably less than 80.

In accordance with the invention the esters used are liquid at ambient temperature (from 20 to 30° C.).

The liquid esters of a C3-C30 carboxylic acid and a C1-C30 alcohol, one at least of the acid or the alcohol being branched or unsaturated, are selected, for example, from esters of a C6-C24 carboxylic acid and a C3-C20 alcohol.

As examples of liquid esters which can be used according to the invention, mention may be made of octyldodecyl behenate; isocetyl behenate; isocetyl lactate; isostearyl lactate; linoleyl lactate; oleyl lactate; isostearyl octanoate; isocetyl octanoate; decyl oleate; isocetyl isotearate; isocetyl laurate; isocetyl stearate; isodecyl octanoate; isodecyl oleate; isononyl isononanoate; isostearyl palmitate; myristyl isostearate; octyl isononanoate; 2-ethylhexyl isononate; octyl isostearate; octyldodecyl erucate; isopropyl palmitates, 2-ethylhexyl palmitate, 2-octyldecyl palmitate, branched alkyl myristates such as isopropyl myristate, t-butyl myristate, 2-octyldodecyl myristate, hexyl isostearate, butyl isostearate, isobutyl stearate; and 2-hexyldecyl laurate.

It is also possible to use the esters of C4-C22 dicarboxylic or tricarboxylic acids and C1-C22 alcohols and the esters of C2-C26 di-, tri-, tetra- or pentahydroxy alcohols and monocarboxylic, dicarboxylic or tricarboxylic acids.

As examples of esters which can be used according to the invention, mention may be made of diisopropyl sebacate; diisopropyl adipate; diisostearyl adipate; octyldodecyl stearoyl stearate; pentaerythrityl tetra-isononanoate; pentaerythrityl tetraisostearate; triisopropyl citrate; triisostearyl citrate; and trioctyldodecyl citrate. Preferably the acid and the alcohol of the ester are saturated.

Among the abovementioned esters it is preferred to use isopropyl palmitate, 2-ethylhexyl palmitate, 2-octyldecyl palmitate, branched alkyl myristates such as isopropyl myristate, t-butyl myristate, and 2-octyldodecyl myristate, hexyl isostearate, butyl isostearate, and isobutyl stearate; 2-hexyldecyl laurate, and isononyl isononanate.

The esters of a C7-C30 aromatic acid and a C1-C30 alcohol are preferably esters of a C7-C17 aromatic acid and a C1-C20 alcohol. These esters are preferably C12-C15 alkyl benzoates, isostearyl benzoate, octyldodecyl benzoate, behenyl benzoate, and 2-ethylhexyl benzoate.

A mixture of all of the abovementioned esters may be used.

In accordance with the invention the carboxylic ester or esters can represent from 0.001% to less than 10% by weight, preferably from 0.01% to 8% by weight, and more preferably still from 0.05% to 6% by weight, of the total weight of the final composition.

The type-1) ester/starch weight ratio is preferably less than or equal to 1.5 and more preferably less than 1. When the composition contains a type-2) ester the ester/starch ratio is preferably less than 2.5 and more preferably less than 2.

In accordance with one preferred embodiment the compositions according to the invention further comprise at least one hair benefit agent such as, silicones, vegetable oils, animal oils, mineral oils or synthetic oils, waxes, ceramides, pseudoceramides, cationic polymers, sunscreens, and vitamins.

The silicones which can be used in accordance with the invention are, for example, polyorganosiloxanes which are insoluble in the composition and can be in the form of oils, waxes, resins or gums.

Organopolysiloxanes are defined in greater detail in the work by Walter NOLL “Chemistry and Technology of Silicones” (1968) Academic Press. They may be volatile or nonvolatile.

When they are volatile, the silicones are more preferably chosen from those having a boiling point of between 60° C. and 260° C., and even more preferably from (i) cyclic silicones or (ii) linear silicones:

cyclic silicones containing from 3 to 7 and preferably 4 to 5 silicon atoms. These are, for example, octamethylcyclotetrasiloxane sold under the name Volatile Silicone 7207 by Union Carbide or Silbione 70045 V 2 by Rhône-Poulenc, decamethylcyclopentasiloxane sold under the name Volatile Silicone 7158 by Union Carbide, and Silbione 70045 V 5 by Rhône-Poulenc, and mixtures thereof.

As examples of cyclic silicones according to the invention, mention may also be made of cyclocopolymers of the dimethylsiloxanes/methylalkylsiloxane type, such as Volatile Silicone FZ 3109 sold by the company Union Carbide, having the chemical structure:

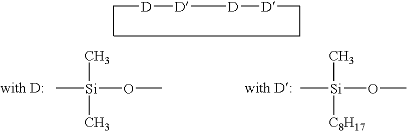

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is the decamethyltetrasiloxane sold under the name SH 200 by the company Toray Silicone. Silicones belonging to this category are also described in the article published in Cosmetics and Toiletries, Vol. 91, January 76, pp. 27-32, Todd & Byers “Volatile Silicone Fluids for Cosmetics”.

Nonvolatile silicones, more preferably, polyalkylsiloxanes, polyarylsiloxanes, polyalkylarylsiloxanes, silicone gums and resins, polyorganosiloxanes modified with organofunctional groups, and mixtures thereof, can also be used according to the invention.

These silicones are more preferably chosen from polyalkylsiloxanes, among which mention may be made mainly of polydimethylsiloxanes containing trimethylsilyl end groups having a viscosity of from 5×10⁻⁶ to 2.5 m²/s at 25° C. and preferably 1×10⁻⁵ to 1 m²/s. The viscosity of the silicones is measured, for example, at 25° C. according to ASTM standard 445 Appendix C.

Among these polyalkylsiloxanes, mention may be made, in a nonlimiting manner, of the following commercial products as examples which can be used according to the invention:
the Silbione oils of the 47 and 70 047 series or the Mirasil oils sold by Rhône-Poulenc, such as, for example, the oil 70 047 V 500 000;
the oils of the Mirasil series sold by the company Rhône-Poulenc;
the oils of the 200 series from the company Dow Corning, such as, more preferably, DC200 with a viscosity of 60 000 cSt;
the Viscasil oils from General Electric and certain oils of the SF series (SF 96, SF 18) from General Electric.

As further examples, mention may also be made of polydimethylsiloxanes containing dimethylsilanol end groups (Dimethiconol according to the CTFA name) such as the oils of the 48 series from the company Rhône-Poulenc.

In this category of polyalkylsiloxanes, mention may also be made of the products sold under the names Abil Wax 9800 and 9801 by the company Goldschmidt, which are poly(C₁-C₂₀)alkylsiloxanes.

The polyalkylarylsiloxanes are chosen preferably from linear and/or branched polydimethylmethylphenylsiloxanes and polydimethyldiphenylsiloxanes with a viscosity of from 1×10⁻⁵ to 5×10⁻² m²/s at 25° C.

Among these polyalkylarylsiloxanes, mention may be made, by way of example, of the products sold under the following names:
the Silbione oils of the 70 641 series from Rhône-Poulenc;
the oils of the Rhodorsil 70 633 and 763 series from Rhône-Poulenc;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

The silicone gums that can be used in accordance with the invention are, preferably, polydiorganosiloxanes having high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane or tridecanes, or mixtures thereof.

Mention may be made, by way of examples, of the following preferred products:
polydimethylsiloxane,
polydimethylsiloxane/methylvinylsiloxane gums,
polydimethylsiloxane/diphenylsiloxane,
polydimethylsiloxane/phenylmethylsiloxane,
polydimethylsiloxane/diphenylsiloxane/methylvinyl-siloxane.

Products that can be used more preferably in accordance with the invention are mixtures such as:
mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs of different viscosities, and more preferably of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product preferably contains 15% SE 30 gum and 85% SF 96 oil.

The organopolysiloxane resins that can be used in accordance with the invention are crosslinked siloxane systems containing the following units: R₂SiO_{2/2}, R₃SiO_{1/2}, RSiO_{3/2} and SiO_{4/2} in which R represents a hydrocarbon-based group containing 1 to 16 carbon atoms or a phenyl group. Among these products, those preferred are the ones in which R denotes a C₁-C₄ lower alkyl radical, more preferably methyl, or a phenyl radical.

Among these resins, mention may be made of the product sold under the name Dow Corning 593 or those sold under the names Silicone Fluid SS 4230 and SS 4267 by the company General Electric, which are silicones of dimethyl/trimethyl siloxane structure.

Mention may also be made, by way of example, of the trimethyl siloxysilicate type resins sold under the names X22-4914, X21-5034 and X21-5037 by the company Shin-Etsu.

The organomodified silicones that can be used in accordance with the invention are silicones as defined above and containing in their structure one or more organofunctional groups attached via a hydrocarbon-based radical.

Among the organomodified silicones, mention may be made of polyorganosiloxanes as examples comprising:
polyethyleneoxy and/or polypropyleneoxy groups optionally comprising C₆-C₂₄ alkyl groups, such as the products known as dimethicone copolyol sold by the company Dow Corning under the name DC 1248 or the oils Silwet L 722, L 7500, L 77 and L 711 by the company Union Carbide, and the (C12)alkylmethicone copolyol sold by the company Dow Corning under the name Q2 5200;
substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, for example, C₁-C₄ aminoalkyl groups;
thiol groups, such as the products sold under the names GP 72 A and GP 71 from Genesee;
alkoxylated groups, such as the product sold under the name Silicone Copolymer F-755 by SWS Silicones and Abil Wax 2428, 2434 and 2440 by the company Goldschmidt;
hydroxylated groups, such as the polyorganosiloxanes containing a hydroxyalkyl function, described in French patent application FR-A-85/16334,corresponding to formula (IX) below: 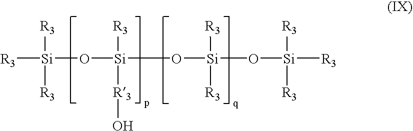
   in which the radicals R₃, which are identical or different, are chosen from methyl or phenyl radicals; at least 60 mol % of the radicals R₃ denoting methyl; the radical R′₃ is a C2-C18 divalent hydrocarbon-based alkylene chain unit; p is between 1 and 30 inclusive; q is between 1 and 150 inclusive;
acyloxyalkyl groups, such as, for example, the polyorganosiloxanes described in patent U.S. Pat. No. 4 957 732 and corresponding to formula (X) below: 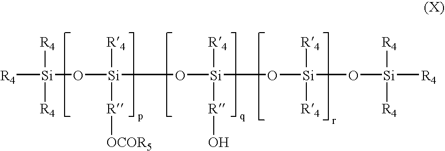
   in which:
   R₄ denotes a methyl, phenyl, —OCOR or hydroxyl group, one of the radicals R₄ per silicon atom possibly being OH;
   R′₄ denotes methyl or phenyl; at least 60 mol % of all the radicals R₄ and R′₄ denoting methyl;
   R₅ denotes C₈-C₂₀ alkyl or alkenyl;
   R″ denotes a C₂-C₁₈ linear or branched divalent hydrocarbon-based alkylene radical;
   r is between 1 and 120 inclusive;
   p is between 1 and 30;
   q is equal to 0 or is less than 0.5 p, p+q being between 1 and 30; the polyorganosiloxanes of formula (VI) may contain the following groups: 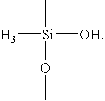
      in proportions not exceeding 15% of the sum p+q+r.
anionic groups of carboxylic type, such as, for example, in the products described in patent EP 186 507 from the company Chisso Corporation, or of alkylcarboxylic type, such as those present in the product X-22-3701E from the company Shin-Etsu; 2-hydroxyalkyl sulfonate; 2-hydroxyalkyl thiosulfate such as the products sold by the company Goldschmidt under the names Abil S201 and Abil S255;
hydroxyacylamino groups, such as the polyorganosiloxanes described in patent application EP 342 834. Mention may be made, for example, of the product Q2-8413 from the company Dow Corning.

According to the invention, it is also possible to use silicones comprising a polysiloxane portion and a portion consisting of a nonsilicone organic chain, one of the two portions constituting the main chain of the polymer, the other being grafted onto said main chain. These polymers are described, for example, in patent applications EP-A-412 704, EP-A-412 707, EP-A-640 105, WO 95/00578, EP-A-582 152 and WO 93/23009 and U.S. Pat. Nos. 4,693,935, 4,728,571 and 4,972,037. These polymers are preferably anionic or nonionic.

Such polymers are, for example, copolymers that can be obtained by free-radical polymerization starting from the monomer mixture consisting of:
a) 50 to 90% by weight of tert-butyl acrylate;
b) 0 to 40% by weight of acrylic acid;
c) 5 to 40% by weight of silicone macromer of the following formula: 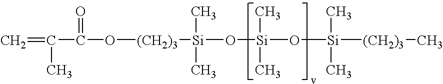
   with v being a number ranging from 5 to 700; the weight percentages being calculated relative to the total weight of the monomers.

Other examples of grafted silicone polymers are, polydimethylsiloxanes (PDMS) onto which are grafted, via a connecting chain unit of thiopropylene type, mixed polymer units of poly(meth)acrylic acid type and of polyalkyl (meth)acrylate type and polydimethylsiloxanes (PDMS) onto which are grafted, via a connecting chain unit of thiopropylene type, polymer units of polyisobutyl (meth)acrylate type.

According to the invention, it is also possible to use polyurethane silicones, such as those described in patent applications EP 0 751 162 and EP 0 619 111.

According to the invention, all of the silicones can also be used in the form of emulsions, nanoemulsions or microemulsions.

The polyorganosiloxanes that are preferred in accordance with the invention are:
nonvolatile silicones selected from the class of polyalkylsiloxanes containing trimethylsilyl end groups, such as oils having a viscosity of between 0.2 and 2.5 m²/s at 25° C., such as the oils of the DC200 series from Dow Corning, preferably that with a viscosity of 60 000 cSt, of the Silbione 70047 and 47 series and more preferably the oil 70 047 V 500 000, which are sold by the company Rhône-Poulenc, polyalkylsiloxanes containing dimethylsilanol end groups, such as dimethiconols, or polyalkylarylsiloxanes such as the oil Silbione 70641 V 200 sold by the company Rhône-Poulenc;
the organopolysiloxane resin sold under the name Dow Corning 593;
polysiloxanes containing amine groups, such as amodimethicones or trimethylsilylamodimethicones.

The cationic polymers which can be used in accordance with the present invention can be selected from all those already known per se as improving the cosmetic properties of the hair, namely those described in patent application EP-A-0 337 354 and in French patent applications FR-A-2 270 846, 2 383 660, 2 598 611, 2 470 596, and 2 519 863.

Even more generally, in the sense of the present invention, the expression “cationic polymer” denotes any polymer containing cationic groups and/or groups which can be ionized to cationic groups.

Preferred cationic polymers are selected from those comprising units containing primary, secondary, tertiary and/or quaternary amine groups which either may form part of the polymer main chain or may be carried by a lateral substituent directly connected to said chain.

The cationic polymers used generally have a number-average molecular mass of between 500 and 5×10⁶ approximately, and preferably between 10³ and 3×10⁶ approximately.

Among the cationic polymers mention may be made more preferably of the polymers of the polyamine, polyaminoamide, and quaternary polyammonium type. These are known products.

Among all the cationic polymers that may be used in the context of the present invention, it is preferred to use quaternary cellulose ether derivatives such as the products sold under the name JR 400 by the company Union Carbide Corporation, cationic cyclopolymers, preferably the dimethyldiallylammonium chloride homopolymers or copolymers sold under the names Merquat 100, Merquat 550 and Merquat S by the company Calgon, and quaternary polymers of vinylpyrrolidone and of vinylimidazole, crosslinked homopolymers or copolymers of methacryloyloxy(C₁-C₄)-alkyltri(C₁-C₄)alkylammonium salts, and mixtures thereof.

According to the invention, at least one hair benefit agent may represent from 0.001% to 20% by weight, preferably from 0.01% to 10% by weight, and more preferably from 0.1 to 5% by weight relative to the total weight of the final composition.

The compositions of the invention further advantageously comprise at least one surfactant, which is generally present in an amount of between 0.01% and 50% by weight approximately, preferably between 0.1% and 40%, and more preferably still between 0.5% and 30%, relative to the total weight of the composition.

Said surfactant may be selected from anionic, amphoteric, nonionic, or cationic surfactants or mixtures thereof.

The surfactants that are suitable for carrying out the present invention are the following:

Anionic Surfactant(s):

In the context of the present invention, their nature does not assume a truly critical character.

Thus, as examples of anionic surfactants which can be used, alone or as mixtures, in the context of the present invention, mention may be made (nonlimiting list) of preferred salts (preferably alkaline salts, such as, sodium salts, ammonium salts, amine salts, amino alcohol salts or magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, the alkyl or acyl radical of all of these various compounds preferably containing from 8 to 24 carbon atoms, and the aryl radical preferably denoting a phenyl or benzyl group. Among the anionic surfactants which can also be used, mention may also be made of fatty acid salts such as the salts of oleic, ricinoleic, palmitic and stearic acids, coconut oil acid or hydrogenated coconut oil acid; acyl lactylates in which the acyl radical contains 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, preferably those containing from 2 to 50 ethylene oxide groups, and mixtures thereof.

Among the anionic surfactants, it is preferred according to the invention to use alkyl sulfate salts and alkyl ether sulfate salts and mixtures thereof.

(ii) Nonionic Surfactant(s):

The nonionic surfactants are, themselves also, compounds that are well known per se (see in this respect “Handbook of Surfactants” by M. R. Porter, published by Blackie & Son (Glasgow and London), 1991, pp. 116-178) and, in the context of the present invention, their nature is not a critical feature. Thus, they can be chosen preferably from (nonlimiting list) polyethoxylated, polypropoxylated or polyglycerolated fatty acids, alkylphenols, alpha-diols or alcohols having a fatty chain containing, for example, 8 to 18 carbon atoms, it being possible for the number of ethylene oxide or propylene oxide groups to range preferably from 2 to 50 and for the number of glycerol groups to range preferably from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and preferably 1.5 to 4, glycerol groups; polyethoxylated fatty amines preferably having 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It will be noted that the alkylpolyglycosides constitute nonionic surfactants that are also suitable in the context of the present invention.

(iii) Amphoteric Surfactant(s):

The amphoteric surfactants, whose nature is not a critical feature in the context of the present invention, can be, for example (nonlimiting list), aliphatic secondary or tertiary amine derivatives in which the aliphatic radical is a linear or branched chain containing 8 to 22 carbon atoms and containing at least one water-soluble anionic group (for example carboxylate, sulfonate, sulfate, phosphate or phosphonate); mention may also be made of (C₈-C₂₀) alkylbetaines, sulfobetaines, (C₈-C₂₀) alkyl-amido (C₁-C₆) alkylbetaines or (C₈-C₂₀) alkylamido (C₁-C₆) alkylsulfobetaines.

As examples of the amine derivatives which can be used according to the invention, mention may be made of the products sold under the name Miranol, as described in U.S. Pat. Nos. 2 528 378 and 2 781 354 and having the structures:
R₂—CONHCH₂CH₂—N(R₃)(R₄)(CH₂COO—) (2)
in which: R₂ denotes an alkyl radical derived from an acid R₂—COOH present in hydrolyzed coconut oil, a heptyl, nonyl or undecyl radical, R₃ denotes a β-hydroxyethyl group and R₄ denotes a carboxymethyl group; and
R₅—CONHCH₂CH₂—N(B)(C) (3)
in which:
B represents —CH₂CH₂OX′, C represents —(CH₂)_{z}—Y′, with z=1 or 2,
X′ denotes the —CH₂CH₂—COOH group or a hydrogen atom,
Y′ denotes —COOH or the —CH₂—CHOH—SO₃H radical,
R5 denotes an alkyl radical of an acid R₉—COOH present in coconut oil or in hydrolyzed linseed oil, an alkyl radical, preferably a C₇, C₉, C₁₁, or C₁₃ alkyl radical, a C₁₇ alkyl radical and its iso form, an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold under the trade name Miranol C2M concentrate by the company Rhône-Poulenc.

(iv) The Cationic Surfactants May be Chosen from:

the quaternary ammonium salts of general formula (IV) below: 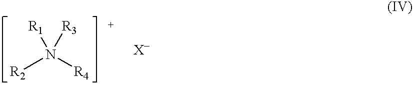
in which X is an anion selected from the group of the halides (chloride, bromide or iodide) or (C₂-C₆)alkyl sulfates, more preferably methyl sulfate, phosphates, alkyl- or alkylaryl-sulfonates, anions derived from organic acid, such as acetate or lactate, and

the radicals R1 to R3, which may be identical or different, represent a linear or branched aliphatic radical containing from 1 to 4 carbon atoms, or an aromatic radical such as aryl or alkylaryl. The aliphatic radicals can contain heteroatoms such as, oxygen, nitrogen, sulfur or halogens. The aliphatic radicals are for example selected from alkyl, alkoxy, and alkylamide radicals, R4 denotes a linear or branched alkyl radical containing from 16 to 30 carbon atoms. The cationic surfactant is preferably a behenyltrimethylammonium salt (for example, chloride).

b) The radicals R1 and R2, which may be identical or different, represent a linear or branched aliphatic radical containing from 1 to 4 carbon atoms, or an aromatic radical such as aryl or alkylaryl. The aliphatic radicals can contain heteroatoms such as, oxygen, nitrogen, sulfur or halogens. The aliphatic radicals are for example selected from alkyl, alkoxy, alkylamide, or hydroxyalkyl radicals, containing approximately from 1 to 4 carbon atoms; R3 and R4, which are identical or different, denote a linear or branched alkyl radical containing from 12 to 30 carbon atoms, said radical comprising at least one ester or amide function. R3 and R4 are selected preferably from (C₁₂-C₂₂)alkylamido(C₂-C₆)alkyl and (C₁₂-C₂₂)alkyl acetate radicals; the cationic surfactant is preferably a stearamidopropyldimethyl( myristyl acetate)ammonium salt (for example, chloride).

B)—The quaternary ammonium salts of imidazolinium, such as, for example, that of formula (V) below: 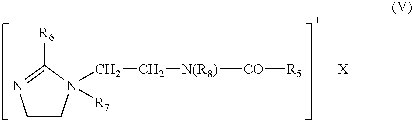
in which R₅ represents an alkyl or alkenyl radical containing from 8 to 30 carbon atoms, for example, fatty acid derivatives of tallow, R₆ represents a hydrogen atom, a C₁-C₄ alkyl radical or an alkyl or alkenyl radical containing from 8 to 30 carbon atoms, R₇ represents a C₁-C₄ alkyl radical, R₈ represents a hydrogen atom or a C₁-C₄ alkyl radical, X is an anion selected from the group of the halides, phosphates, acetates, lactates, alkyl sulfates, alkyl- or alkylaryl-sulfonates. Preferably R₅ and R₆ denote a mixture of alkyl or alkenyl radicals containing from to 21 carbon atoms, for example fatty acid derivatives of tallow, R₇ denotes methyl, R₈ denotes hydrogen. Such a product is, for example, Quaternium-27 (CTFA 1997) or Quaternium-83 (CTFA 1997), which are sold under the names “REWOQUAT” W 75, W90, W75PG, and W75HPG by the company WITCO,

C)—the quaternary diammonium salts of formula (VI) below: 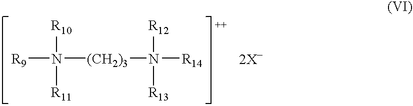
in which R₉ denotes an aliphatic radical containing approximately from 16 to 30 carbon atoms, R₁₀, R₁₁, R₁₂, R₁₃, and R₁₄, identical or different, are selected from hydrogen or an alkyl radical containing from 1 to 4 carbon atoms, and X is an anion selected from the group of the halides, acetates, phosphates, nitrates, or methyl sulfates. Quaternary diammonium salts of this kind include preferably propanetallowdiammonium dichloride.

D)—The quaternary ammonium salts containing at least one ester function, of formula (VII) below: 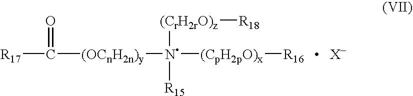
in which:
R₁₅ is selected from C₁-C₆ alkyl radicals and C₁-C₆ hydroxyalkyl or dihydroxyalkyl radicals;
R₁₆ is selected from:
   the radical 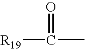
   linear or branched, saturated or unsaturated C₁-C₂₂ hydrocarbon radicals R₂₀,
   the hydrogen atom,
R₁₈ is selected from:
   the radical 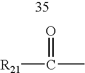
   linear or branched, saturated or unsaturated C₁-C₆ hydrocarbon radicals R₂₂,
   the hydrogen atom,
R₁₇, R₁₉, and R₂₁, identical or different, are selected from linear or branched, saturated or unsaturated C₇-C₂₁ hydrocarbon radicals;
n, p, and r, identical or different, are integers with a value of from 2 to 6;
y is an integer with a value of from 1 to 10;
x and z, identical or different, are integers with a value of from 0 to 10;
X⁻ is a simple or complex, organic or inorganic anion; with the proviso that the sum x+y+z is from 1 to 15 in value, that when x is 0 then R₁₆ denotes R₂₀, and when z is 0 then R₁₈ denotes R₂₂.

The ammonium salts of formula (VII) is more preferably used, in which:
R₁₅ denotes a methyl or ethyl radical,
x and y are equal to 1;
z is equal to 0 or 1;
n, p, and r equal to 2;
R₁₆ is selected from:
   the radical 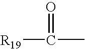
   methyl, ethyl or C₁₄-C₂₂ hydrocarbon radicals
   the hydrogen atom;
R₁₇, R₁₉ and R₂₁, identical or different, are selected from linear or branched, saturated or unsaturated C₇-C₂₁ hydrocarbon radicals;
R₁₈ is selected from:
   the radical 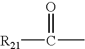
   the hydrogen atom;
      Compounds of this kind are sold for example under the names DEHYQUART by the company HENKEL, STEPANQUAT by the company STEPAN, NOXAMIUM by the company CECA, and REWOQUAT WE 18 by the company REWO-WITCO.

Among the quaternary ammonium salts preference is given to behenyltrimethylammonium chloride or else the stearamidopropyldimethyl(myristyl acetate)ammonium chloride sold under the name CERAPHYL 70 by the company VAN DYK, Quaternium-27 or Quaternium-83, which are sold by the company WITCO.

The composition comprises preferably from 30 to 99% of water, preferably from 50 to 95% of water, and more preferably still from 70 to 95% by weight of water, relative to the total weight of the composition.

The cosmetically acceptable aqueous medium comprises water alone or a mixture of water and a cosmetically acceptable solvent selected from C₁-C₄ lower alcohols, such as ethanol, isopropanol, tertbutanol or n-butanol; polyols such as propylene glycol; polyol ethers; C₅-C₁₀ alkanes; C₃-C₄ ketones such as acetone and methyl ethyl ketone; C₁-C₄ alkyl acetates such as methyl acetate, ethyl acetate, and butyl acetate; dimethoxyethane and diethoxyethane; and mixtures thereof.

The pH of the compositions of the invention is between 4 and 8, preferably between 5 and 7.

The composition of the invention may also comprise at least one additive chosen from thickeners, fragrances, nacreous agents, preservatives, silicone or nonsilicone sunscreens, vitamins, provitamins, anionic or nonionic polymers, proteins, protein hydrolyzates, 18-methyleicosanoic acid, hydroxy acids, vitamins, provitamins such as panthenol, and any other additive conventionally used in cosmetology that does not affect the properties of the compositions according to the invention.

The skilled worker will take care to select any additives and their amount such that they are not detrimental to the properties of the compositions of the present invention.

These additives are present where appropriate in the composition according to the invention in proportions that may range from 0.001 to 20% by weight relative to the total weight of the composition. The precise amount of each additive is readily determined by the skilled worker, depending on its nature and its function.

The compositions in accordance with the invention may be used more preferably for washing or treating keratin materials such as the hair, the skin, the eyelashes, the eyebrows, the nails, the lips or the scalp, and more preferably the hair.

The compositions of the invention may be in the form of a rinse-out or leave-in conditioner, perming, hair-straightening, coloring or bleaching compositions, or alternatively in the form of rinse-out compositions to be applied before or after coloring, bleaching, perming or hairstraightening, or else between the two steps of a perming or hair-straightening operation.

In one preferred embodiment of the invention the composition can be used as a conditioner. When the composition is in the form of a conditioner or a care composition which, where appropriate, is to be rinsed out, it advantageously includes at least one cationic surfactant, the concentration thereof being generally between 0.1% and 10% by weight and preferably from 0.5% to 5% by weight relative to the total weight of the composition.

It may also comprise one or more amphoteric or nonionic surfactants.

The compositions according to the invention may be detergent compositions such as shampoos, shower gels, and bubble baths. In this embodiment of the invention the compositions comprise at least one washing base, which is generally aqueous.

The surfactant(s) constituting the washing base may be selected, without discrimination, alone or in mixtures, from the anionic, amphoteric, nonionic, and cationic surfactants as defined above. The washing base comprises at least one detergent surfactant.

It is preferred to use an anionic surfactant selected from sodium, triethanolamine or ammonium (C₁₂-C₁₄)alkyl sulfates, sodium, triethanolamine or ammonium (C₁₂-C₁₄)alkyl ether sulfates ethoxylated with 2.2 moles of ethylene oxide, sodium cocoyl isethionate, or sodium alpha-(C₁₄-C₁₆)olefinsulfonate, and mixtures thereof, with:

either an amphoteric surfactant such as the amine derivatives known as disodium cocoamphodipropionate or sodium cocoamphopropionate, sold by the company Rhône-Poulenc under the trade name Miranol C2M CONC as an aqueous solution with an active substance content of 38% or under the name Miranol C32;

or an amphoteric surfactant of zwitterionic type such as alkylbetaines, preferably the cocobetaine sold under the name Dehyton AB 30 as an aqueous solution containing 32% AS by the company Henkel.

The quantity and quality of the washing base are those which are sufficient to give the final composition a satisfactory foaming and/or detergent power.

Thus in accordance with the invention the washing base can represent from 4% to 50% by weight, preferably from 6% to 35% by weight and more preferably still from 8% to 25% by weight, of the total weight of the final composition.

The invention further provides a process for treating keratin materials such as the skin or the hair, characterized in that it consists in applying to the keratin materials a cosmetic composition as defined above, optionally followed by rinsing with water.

Thus, this process according to the invention allows hold of the hairstyle, the treatment, care, washing or removal of makeup from the skin, the hair or any other keratin material, and more preferably the hair.

The compositions of the invention may also be in the form of washing compositions for the skin, and preferably in the form of bath or shower solutions or gels or makeup remover products.

The compositions according to the invention may also be in the form of aqueous or aqueous-alcoholic lotions for skincare and/or haircare.

The cosmetic compositions according to the invention may be in the form of a gel, a milk, a cream, an emulsion, a thickened lotion or a mousse and may be used for the skin, the nails, the eyelashes, the lips and, more preferably, the hair.

The compositions may be packaged in various forms, such as in vaporizers, pump-dispenser bottles or in aerosol containers to allow the composition to be applied in vaporized form or in the form of a mousse. Such packaging forms are indicated, for example, when it is desired to obtain a spray, a lacquer or a mousse for treating the hair.

Throughout the text hereinabove and hereinbelow, the percentages expressed are on a weight basis.

The invention will now be illustrated more fully with the aid of the examples that follow, which should not be considered as limiting it to the embodiments described.

### EXAMPLE 1

A conditioner in accordance with the invention was produced, with the following composition:

| | | | |
|---|---|---|---|
| Pregelatinized corn distarch phosphate | | 5.3 | g AS |
| (STRUCTURE ZEA from NATIONAL STARCH) | | | |
| Isopropyl myristate | | 1 | g |
| Behenyltrimethylammonium chloride | | 3.2 | g AS |
| Cetyl stearyl alcohol (50/50 by weight) | | 4 | g |
| Water | qs | 100 | g |

The composition has a gelatinous and highly melting texture when applied to wet hair. Its rinsability is good. The wet hair is not heavy and hair shaping is easy.

### EXAMPLE 2

A conditioner in accordance with the invention was produced, with the following composition:

| | | | |
|---|---|---|---|
| Potato fecula modified with | | 1.5 | g |
| 2-chloro-ethylamidodipropionic | | | |
| acid neutralized with sodium | | | |
| hydroxide (Solanace Starch | | | |
| from NATIONAL STARCH) | | | |
| Isononyl isononanoate | | 1 | g |
| Quaternium-87 (REWOQUAT WPG | | 3.75 | g AS |
| 75 from WITCO) | | | |
| Cetyl stearyl alcohol (50/50 by weight) | | 4 | g |
| Water | qs | 100 | g |

The treated hair has the same properties as that treated with the composition of Example 1.

### EXAMPLE 3

A conditioner in accordance with the invention was produced, with the following composition:

| | | | |
|---|---|---|---|
| Potato fecula modified with | | 3 | g |
| 2-chloro-ethylamidodipropionic | | | |
| acid neutralized with sodium | | | |
| hydroxide (Solanace Starch | | | |
| from NATIONAL STARCH) | | | |
| C12-C15 alkyl benzoate | | 5 | g |
| (FINSOLV TN from GOLDSCHMIDT) | | | |
| Benzophenone-3 (UVINUL M40 from BASF) | | 1 | g |
| Isopropyl myristate | | 0.8 | g |
| Quaternium-87 (REWOQUAT WPG | | 3.75 | g AS |
| 75 from WITCO) | | | |
| Cetyl stearyl alcohol (50/50 by weight) | | 4 | g |
| Water | qs | 100 | g |

The treated hair has the same properties as that treated with the composition of Example 1.

### EXAMPLE 4

A conditioner in accordance with the invention was produced, with the following composition:

| | | | |
|---|---|---|---|
| Potato fecula modified with | | 3 | g |
| 2-chloroethylamidodipropionic | | | |
| acid neutralized with sodium | | | |
| hydroxide (Solanace Starch | | | |
| from NATIONAL STARCH) | | | |
| Isopropyl myristate | | 0.6 | g |
| Mixture of cetyl stearyl alcohol | | 5 | g |
| (50/50 by weight) and ethoxylated | | | |
| cetyl stearyl alcohol (30 EO) | | | |
| (SINNOWAX AO from COGNIS) | | | |
| Water | qs | 100 | g |

The treated hair has the same properties as that treated with the composition of Example 1.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition, comprising:
a cosmetically acceptable medium,
at least one starch,
at least one carboxylic ester in an amount of up to 10% based on the weight of said cosmetic composition wherein said carboxylic ester is selected from
1) liquid esters of a C3-C30 carboxylic acid and a C1-C30 alcohol, wherein at least one of said acid or said alcohol is branched or possesses at least one carbon-carbon double bond,
2) esters of a C7-C30 aromatic acid and a C1-C30 alcohol, wherein said aromatic acid has carboxyl group directly bonded to said aromatic acid's ring;
at least 30% by weight of water relative to the total weight of said composition, and
up to 20% by weight of fatty phase relative to the total weight of said composition,
with the provisos that
said cosmetic composition is substantially free from fatty acid soap,
said cosmetic composition is free of nonsilicone cationic polymer if said starch is a starch phosphate, and
said carboxylic ester is not isopropyl palmitate or diisopropyl adipate, if said cosmetic composition further comprises at least one thickener and less than 3% by weight of surfactant relative to the weight of the composition.

2. The cosmetic composition of claim 1, wherein said starch is a corn starch, rice starch, manioc starch, tapioca starch, barley starch, potato starch, wheat starch, sorghum starch, or pea starch.

3. The cosmetic composition of claim 1, wherein said starch is modified by one or more of the following reactions.

4. The cosmetic composition of claim 1, wherein said starch is monostarch phosphates, distarch phosphates, tristarch phosphate or a mixture thereof.

5. The cosmetic composition of claim 1, wherein said starch is an amphoteric starch.

6. The cosmetic composition of claim 1, wherein said carboxylic ester is octyldodecyl behenate; isocetyl behenate; isocetyl lactate; isostearyl lactate; linoleyl lactate; oleyl lactate; isostearyl octanoate; isocetyl octanoate; decyl oleate; isocetyl isotearate; isocetyl laurate; isocetyl stearate; isodecyl octanoate; isodecyl oleate; isononyl isononanoate; isostearyl palmitate; myristyl isostearate; octyl isononanoate; 2-ethylhexyl isononate; octyl isostearate; octyldodecyl erucate; isopropyl palmitates, 2-ethylhexyl palmitate, 2-octyldecyl palmitate, branched alkyl myristates such as isopropyl myristate, t-butyl myristate, 2-octyldodecyl myristate, hexyl isostearate, butyl isostearate, isobutyl stearate; 2-hexyldecyl laurate; diisopropyl sebacate; diisopropyl adipate; diisostearyl adipate; octyldodecyl stearoyl stearate; pentaerythrityl tetraisononanoate; pentaerythrityl tetraisostearate; triisopropyl citrate; triisostearyl citrate; or trioctyldodecyl citrate.

7. The cosmetic composition of claim 6, wherein said carboxylic ester is isopropyl palmitate, 2-ethylhexyl palmitate, 2-octyldecyl palmitate, branched alkyl myristates such as isopropyl myristate, t-butyl myristate, and 2-octyldodecyl myristate, hexyl isostearate, butyl isostearate, isobutyl stearate; 2-hexyldecyl laurate, or isononyl isononanate.

8. The cosmetic composition according of claim 1, wherein said carboxylic ester is a C12-C15 alkyl benzoate, isostearyl benzoate, octyldodecyl benzoate, behenyl benzoate, or 2-ethylhexyl benzoate.

9. The cosmetic composition of claim 1, further comprising at least one hair benefit agent.

10. The cosmetic composition of claim 1, wherein said starch is present at a concentration ranging from 0.01% to 20% by weight relative to the total weight of the composition.

11. The cosmetic composition of claim 1, wherein said carboxylic ester is present at a concentration ranging from 0.001% to less than 10% by weight relative to the total weight of the composition.

12. The cosmetic composition of claim 9, wherein said hair benefit agent is present at a concentration ranging from 0.001% to 20% by weight relative to the total weight of the composition.

13. The cosmetic composition of claim 9, further comprising at least one surfactant selected from an anionic, nonionic, amphoteric, cationic surfactant or a mixtures thereof.

14. The cosmetic composition of claim 9, further comprising a cationic surfactant or a mixtures thereof.

15. The cosmetic composition of claim 13, wherein said surfactant is present at a concentration from 0.01% to 50% by weight relative to the total weight of the composition.

16. The cosmetic composition of claim 1, wherein said cosmetic composition is a shampoo, conditioner, composition for perming, straightening, coloring or bleaching the hair, rinse-off composition to be applied between the two steps of a perming or hair-straightening operation, styling gel, styling spray, styling mousse or washing composition for the body.

17. A method of washing or caring for keratinous materials by applying said cosmetic composition according to claim 1 to keratinous materials.

18. A method of treating or conditioning keratinous materials by applying said cosmetic composition according to claim 1 to keratinous materials.

19. The method of washing and caring keratinous materials of claim 17, wherein said cosmetic composition is a shampoo, conditioner, composition for perming, straightening, coloring or bleaching the hair, rinse-off composition to be applied between the two steps of perming or hair-straightening operation, gel, spray, mousse, lotion, cream emulsion or washing composition for body.

20. The method of washing or caring keratinous materials of claim 17, further comprising rinsing said cosmetic composition with water.

21. The method of washing and caring keratinous materials of claim 18, wherein said cosmetic composition is a shampoo, conditioner, composition for perming, straightening, coloring or bleaching the hair, rinse-off composition to be applied between the two steps of perming or hair-straightening operation, gel, spray, mousse, lotion, cream emulsion or washing composition for body.

22. The method of washing or caring keratinous materials of claim 18, further comprising rinsing said cosmetic composition with water.

23. The cosmetic composition of claim 1, wherein said at least one starch is a plurality of starches.

24. The cosmetic composition of claim 10, wherein said starch is present at a concentration ranging from 0.05% to 15% by weight relative to the total weight of the composition.

25. The cosmetic composition of claim 24, wherein said starch is present at a concentration ranging from 0.1% to 10% by weight relative to the total weight of the composition.

26. The cosmetic composition of claim 1, wherein said at least one carboxylic ester is a plurality of carboxylic esters.

27. The cosmetic composition of claim 11, wherein said carboxylic ester is present at a concentration ranging from 0.01% to 8% by weight relative to the total weight of the composition.

28. The cosmetic composition of claim 27, wherein said carboxylic ester is present at a concentration ranging from 0.05% to 6% by weight relative to the total weight of the composition.

29. The cosmetic composition of claim 9, wherein said at least one hair benefit agent is a plurality hair benefit agents.

30. The cosmetic composition of claim 12, wherein said hair benefit agent is present at a concentration ranging from 0.01% to 10% by weight relative to the total weight of the composition.

31. The cosmetic composition of claim 1, further comprising at least one surfactant selected from an anionic, nonionic, amphoteric, cationic surfactant or a mixture thereof.

32. The cosmetic composition according to claim 13, wherein said at least one surfactant is a plurality of surfactants.

33. The cosmetic composition of claim 31, wherein said at least one surfactant is a plurality of surfactants.

34. The cosmetic composition of claim 15, wherein said surfactant is present at a concentration ranging from 0.1% to 40% by weight relative to the total weight of the composition.

35. The cosmetic composition of claim 34, wherein said surfactant is present at a concentration ranging from 0.5% to 30% by weight relative to the total weight of the composition.

36. The cosmetic composition of claim 31, wherein said surfactant is present at a concentration ranging from 0.01% to 50% by weight relative to the total weight of the composition.

37. The cosmetic composition of claim 36, wherein said surfactant is present at a concentration ranging from 0.1% to 40% by weight relative to the total weight of the composition.

38. The cosmetic composition of claim 35, wherein said surfactant is present at a concentration ranging from 0.5% to 30% by weight relative to the total weight of the composition.

39. The cosmetic composition of claim 3, wherein said reaction is pregelatinization, oxidation, crosslinking, esterification or thermal treatment.

40. The cosmetic composition of claim 9, wherein said hair benefit agent is a vegetable oil, animal oil, mineral oil, synthetic oil, wax, ceramide, psedoceramide, silicone, cationic polymer, sunscreen or vitamin.

41. The method of washing or caring for keratinous materials of claim 17, wherein said cosmetic composition further comprises at least one hair benefit agent.

42. The method of washing or caring for keratinous materials of claim 41, wherein said cosmetic composition further comprises at least one surfactant.

43. The method of treating or conditioning keratinous materials of claim 18, wherein said cosmetic composition further comprises at least one hair benefit agent.

44. The method of treating or conditioning keratinous materials of claim 43, wherein said cosmetic composition further comprises at least one surfactant.

45. A method of washing or caring keratinous materials by applying said cosmetic composition of claim 9 to keratinous materials.

46. A method of washing or caring keratinous materials by applying said cosmetic composition of claim 13 to keratinous materials.

47. A method of treating or conditioning keratinous materials by applying said cosmetic composition of claim 9 to keratinous materials.

48. A method of treating or conditioning keratinous materials by applying said cosmetic composition of claim 13 to keratinous materials.

49. The cosmetic composition of claim 1, wherein a ratio of said liquid ester of a C3-C30 carboxylic acid and a C1-C30 alcohol to said starch is less than or equal to 1.5.

50. The cosmetic composition of claim 49, wherein said ratio is less than 1.

51. The cosmetic composition of claim 1, wherein a ratio of said ester of a C7-C30 aromatic acid and a C1-C30 alcohol to said starch is less than 2.5.

52. The cosmetic composition of claim 51, wherein said ratio is less than 2.

53. The cosmetic composition of claim 1, wherein said cosmetic composition has a pH of between 4 and 8.

54. The cosmetic composition of claim 53, wherein said pH is between 5 and 7.

55. The cosmetic composition of claim 13, further comprising at least one additive.

56. The cosmetic composition of claim 55, wherein said additive is a thickener, fragrance nacreous agent, preservative, silicone, nonsilicone sunscreen, vitamin, provitamin, anionic or nonionic polymer, protein, protein hydrolyzate, 18-methyleicosanoic acid, hydroxy acid, or panthenol.

57. The cosmetic composition of claim 56, wherein said additive is present at a concentration ranging from 0.001% to 20% by weight relative to the total weight of said cosmetic composition.

58. The cosmetic composition of claim 1, wherein said carboxylic ester is not isopropyl palmitate or diisopropyl adipate if said cosmetic composition further comprises a phosphorylated starch derivative, at least one thickener, and less than 3% by weight of surfactant relative to the weight of the composition.
